(19) United States
(12) Patent Application Publication
(10) Pub. No.: US 2025/0176941 A1
(43) Pub. Date: Jun. 5, 2025

(54) METHOD AND SYSTEM FOR PROVIDING A CONTINUOUS GUIDANCE USER INTERFACE FOR ACQUIRING A TARGET VIEW OF AN ULTRASOUND IMAGE

(71) Applicant: GE Precision Healthcare LLC, Waukesha, WI (US)

(72) Inventors: Doron Shaked, Tivon (IL); Alexander Sokulin, Kiryat Tivon (IL); Menachem Halmann, Monona, WI (US); Carmit Shiran, Middleton, WI (US); Lev Greenberg, Haifa (IL); Radhika Madhavan, Latham, NY (US)

(21) Appl. No.: 18/529,481

(22) Filed: Dec. 5, 2023

(51) Int. Cl. A61B 8/00; A61B 8/08

(52) U.S. Cl. CPC A61B 8/463; A61B 8/4245; A61B 8/483

(57) ABSTRACT

Systems and methods for acquiring a target ultrasound image comprising acquiring an ultrasound image, processing the ultrasound image to identify a location of the ultrasound probe relative to the target view, presenting the ultrasound image and the reference target view image, wherein the ultrasound image is presented at a first position relative to a second position on the display system of the reference target view image, and wherein the first position and the second position provide feedback for moving the ultrasound probe to acquire the target ultrasound image having the target view, tracking the location of the ultrasound probe relative to the target view in one or more subsequently acquired ultrasound images, and updating the first position on the display system based on the location of the ultrasound probe.

### FIELD

Certain embodiments relate to ultrasound imaging. More specifically, certain embodiments relate to a method and system for providing a continuous guidance user interface for acquiring a target view in ultrasound imaging.

### BACKGROUND

Ultrasound imaging is a medical imaging technique for imaging anatomical structures, such as organs and soft tissues, in a human body. Ultrasound imaging uses real time, non-invasive high frequency sound waves to produce two-dimensional (2D), three-dimensional (3D), and/or four-dimensional (4D) (i.e., real-time/continuous 3D images) images.

Ultrasound imaging is a powerful tool for visualization. Ultrasound images are acquired by an ultrasound probe that may be guided in order to capture a target view of a structure. However, current methods and ultrasound systems for acquiring a target view of structures are complicated (i.e., many manual steps), do not provide feedback to the user, and require a great sense of space and familiarity with an ultrasound scanner.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for a continuous guidance user interface for acquiring a target view of an ultrasound image, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to provide a continuous guidance user interface for acquiring a target view of an anatomical structure, in accordance with various embodiments.

FIG. 2 is an exemplary display of a user interface for acquiring a target view of a target structure, in accordance with various embodiments.

FIG. 3 is a second exemplary display of a user interface for acquiring a target view of a target structure, in accordance with various embodiments.

FIG. 4 is a third exemplary display of a user interface for acquiring a target view of a target structure, in accordance with various embodiments.

FIG. 5 is a fourth exemplary display of a user interface for acquiring a target view of a target structure, in accordance with various embodiments.

FIG. 6 is a fifth exemplary display of a user interface for acquiring a target view of a target structure, in accordance with various embodiments.

FIG. 7 is a flow chart illustrating exemplary steps 802-816 that may be utilized for providing a continuous guidance user interface for acquiring a target view of a target structure, in accordance with various embodiments.

### DETAILED DESCRIPTION

Certain embodiments may be found in a method and system for acquiring a target view of an ultrasound image. Aspects of the present disclosure have the technical effect of providing real-time and continuous feedback regarding movement of an ultrasound probe in order to guide the user to a target view for an ultrasound scan. Various embodiments have the technical effect of presenting an ultrasound operator with the position of an ultrasound probe relative to a target view for an anatomical structure and for providing continuous feedback as the ultrasound probe is moved towards a target view of a target anatomical structure. Certain embodiments have the technical effect of presenting an ultrasound operator with an intuitive user interface that provides customized feedback for acquiring a target view. Aspects of the present disclosure have the technical effect of providing a user with a user interface including an ultrasound image and a target view simultaneously viewable on a display.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical, and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode, which can be one-dimensional (1D), two-dimensional (2D), three-dimensional (3D), or four-dimensional (4D), and comprising Brightness mode (B-mode), Motion mode (M-mode), Color Motion mode (CM-mode), Color Flow mode (CF-mode), Pulsed Wave (PW) Doppler, Continuous Wave (CW) Doppler, Contrast Enhanced Ultrasound (CEUS), and/or sub-modes of B-mode and/or CF-mode such as Harmonic Imaging, Shear Wave Elasticity Imaging (SWEI), Strain Elastography, Tissue Velocity Imaging (TVI), Power Doppler Imaging (PDI), B-flow, Micro Vascular Imaging (MVI), Ultrasound-Guided Attenuation Parameter (UGAP), and the like. The term, “ultrasound image,” as used herein, is used to refer to ultrasound image and/or ultrasound image volumes, such as a bi-plane image, a single 2D image, a rendering of a volume (3D/4D), 2D biplane image slices extracted from a volume (3D/4D), and/or any suitable ultrasound images.

Furthermore, the term processor or processing unit, as used herein, refers to any type of processing unit that can carry out the required calculations needed for the various embodiments, such as single or multi-core: CPU, Accelerated Processing Unit (APU), Graphic Processing Unit (GPU), Digital Signal Processor (DSP), Field-Programmable Gate Array (FPGA), Application-Specific Integrated Circuit (ASIC), or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. In addition, forming of images may be performed using channel combinations that may originate from more than one transmit event (e.g., synthetic aperture techniques).

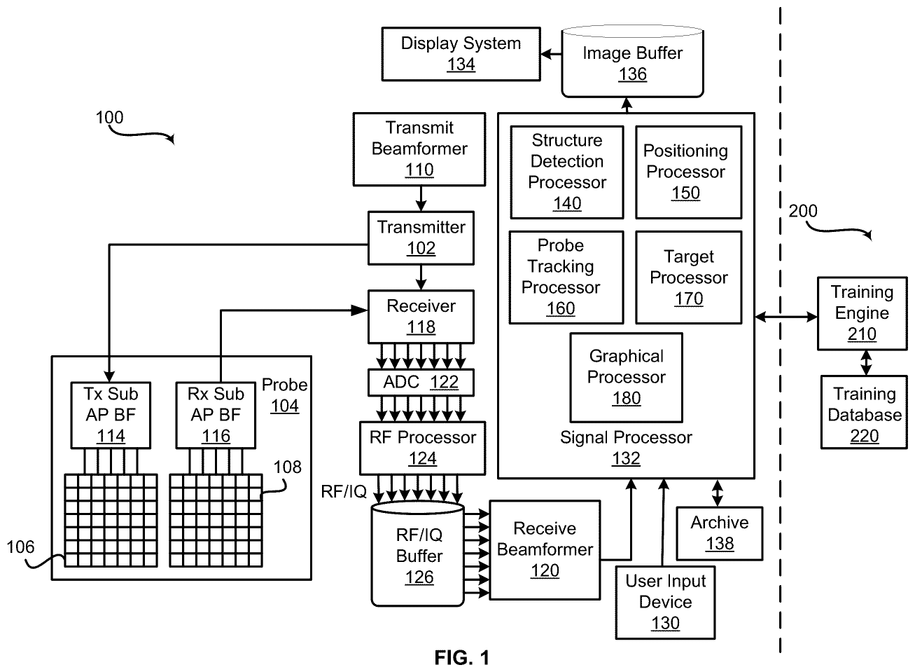

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to acquire a target view of an anatomical structure using real-time feedback, in accordance with various embodiments. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, analog-to-digital (A/D) converters 122, a radio frequency (RF) processor 124, a RF quadrature (RF/IQ) buffer 126, a user input device 130, a signal processor 132, an image buffer 136, a display system 134, and an archive 138.

The transmitter 102 may comprise suitable logic, circuitry, interfaces, and/or code that may be operable to drive an ultrasound probe 104. The ultrasound probe 104 may comprise a two-dimensional (2D) array of piezoelectric elements. In various embodiments, the ultrasound probe 104 may be a matrix array transducer or any suitable transducer operable to acquire 2D and/or 3D (including 4D) ultrasound image datasets. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as an abdomen, a heart, a fetus, a lung, a blood vessel, or any suitable anatomical structure(s).

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces, and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces, and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces, and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select a target view of a target structure, select a reference target view image of anatomical structures, and the like. In an exemplary embodiment, the user input device 130 may be operable to configure, manage, and/or control operation of one or more components and/or modules in the ultrasound system 100. In this regard, the user input device 130 may be operable to configure, manage, and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera, and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), a remote archive, or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise an image acquisition processor 140, a positioning processor 150, a probe tracking processor 160, a target processor 170, and a graphical processor 180. The signal processor 132 may be capable of receiving input information from a user input device 130 and/or archive 138, generating an output displayable by a display system 134, and manipulating the output in response to input information from a user input device 130, among other things. The signal processor 132, image acquisition processor 140, positioning processor 150, probe tracking processor 160, target processor 170, and graphical processor 180 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include an image acquisition processor 140 that comprises suitable logic, circuitry, interfaces and/or code that may be operable to acquire ultrasound images in order to determine a location of the ultrasound probe 104 relative to a target view of a target structure. For example, the image acquisition processor 140 may obtain an ultrasound image and/or consecutive ultrasound images as an ultrasound probe 104 moves towards a target structure in order to obtain a target view of the target structure. In some examples, the ultrasound images obtained may be provided to a positioning processor 150, a probe tracking processor 160, target processor 170, and/or a graphical processor 180. Additionally and/or alternatively, the ultrasound images may be stored in an archive 138.

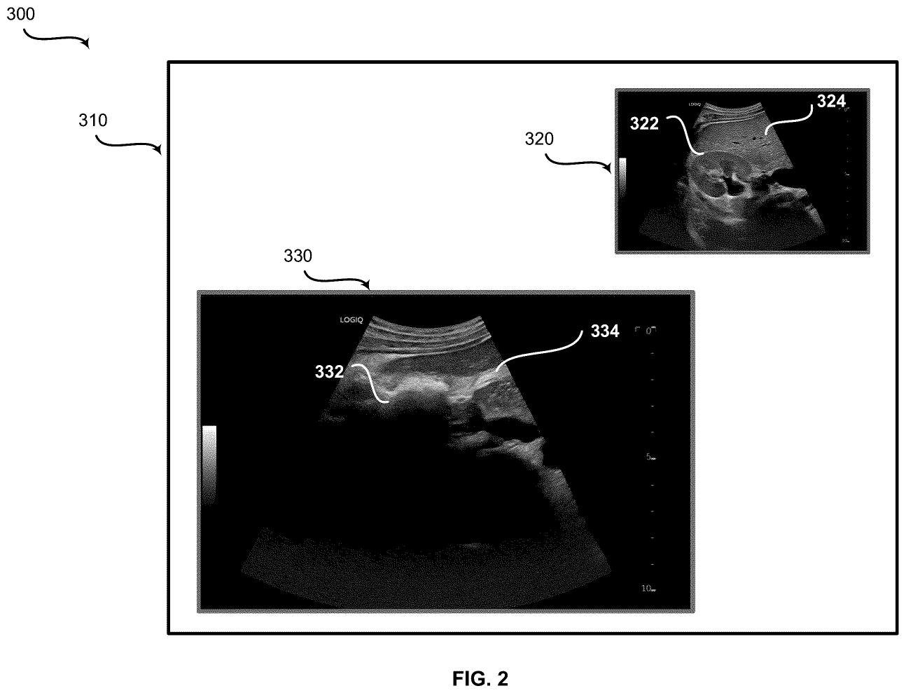

FIG. 2 is an exemplary display 300 of a user interface 310 for acquiring a target view of an anatomical structure, in accordance with various embodiments. Referring to FIG. 2, the display 300 includes a user interface 310 including a reference target view image 320 having a target view 324 of a target structure 322 and an ultrasound image 330 having a target view 334. The ultrasound image 330 may represent an ultrasound probe 104 location relative to the reference target view image 320 on the display 300, which is representative of an ultrasound probe 104 relative to a target structure 322. Although FIG. 2 depicts an ultrasound image 330 representing the probe location, the probe location may be a pointer, an illustration, animation, a graphical marker, or other designator, as non-limiting examples.

The user interface 310 may be provided on a main display of the display system 134 with the user interface 310 as shown in FIG. 2 and/or may be provided on a touch panel of the display system 134. The user interface 310 may provide a user with options for one or more anatomical structures that the user may select. For example, in response to the image acquisition processor 140 causing the display system 134 to present the ultrasound image 330, an ultrasound operator may provide an input via the user input device 130 and/or touchscreen display 130, 134 to select one or more of the anatomical structures as a target structure.

The image acquisition processor 140 may be configured to receive a user input selecting a target structure. The user interface 310 may provide a user a number of reference ultrasound images that include different views for the selected target structure (e.g. different angles, different rotations, different zoom levels, different portions of the structure, etc.). For example, the user interface 310 may provide options to select a target structure and a target view for the target structure, such as “Longitudinal Kidney view”, “Median Transverse Kidney view”, or “Main Portal Vein Entering the Liver view,” as non-limiting examples. The reference ultrasound images and/or reference target views may be obtained from the archive 138. The user may select a reference target view of the reference ultrasound images provided, which may be displayed as the reference target view image 320 on the user interface 310. The reference target view image 320 may include a target view 324 that is helpful in providing a user with a view from a certain angle, position, rotation, and/or zoom level in order to better view a certain area of the selected structure to provide a diagnosis, and/or to obtain an ultrasound image with the target view of the target structure. In some embodiments, an ultrasound operator may provide an input via the user input device 130 and/or touchscreen display 130, 134 to define and/or modify a target view of the selected target structure. In some embodiments, the positioning processor 150 may provide an input to select a different target view of the selected target structure, and/or select a different target structure.

The user input selecting the reference target view image 320 may trigger ultrasound image acquisitions by the ultrasound probe 104 and/or the user may initiate continuous acquisition of ultrasound images, which may displayed as ultrasound image 330 in the user interface 310. The ultrasound image 330 may be a 2D image or a volume acquisition. The ultrasound image 330 may be provided by the image acquisition processor 140 to a positioning processor 150, probe tracking processor 160, target processor 170, and/or graphical processor 180. Additionally and/or alternatively, the ultrasound image 330 may be stored at archive 138 and/or any suitable computer readable medium.

Referring again to FIG. 1, the image acquisition processor 140 may be configured to acquire ultrasound images as the ultrasound probe 104 moves towards a reference target view image 320 of the selected target structure as described above. As the user moves the ultrasound probe 104 towards the target structure, the ultrasound probe may capture ultrasound images and provide the updated images to the positioning processor 150 and/or update the ultrasound image 330. In some embodiments, the image acquisition processor 140 may be configured to analyze ultrasound images acquired to determine a location and/or view of the ultrasound probe 104 as the ultrasound probe 104 moves towards the target structure. The acquired ultrasound images may be provided by the image acquisition processor 140 to the positioning processor 150, the target processor 170, and/or the graphical processor 180. Additionally and/or alternatively, the acquired ultrasound images may be stored at archive 138 and/or any suitable computer readable medium and/or may be displayed on the display 300. In an exemplary embodiment, the image acquisition processor 140 may automatically initiate subsequent ultrasound image acquisitions by the ultrasound probe 104 on the selected target structure 432 if a user input is not received within a pre-defined time period. The image acquisition processor 140 may be configured to initiate the second ultrasound image acquisitions by the ultrasound probe 104 in response to a user input. In addition, the image acquisition processor 140 may be configured to initiate the subsequent ultrasound image acquisitions by the ultrasound probe 104 in response to an expiration of a pre-defined period of time after the previous image acquisition or after the last user input. In some embodiments, the subsequent image acquisitions may be continuous, and/or real-time to reflect movement of the ultrasound image 430 representing the ultrasound probe 104 location towards the active view 420.

The signal processor 132 may include a positioning processor 150 that comprises suitable logic, circuitry, interfaces, and/or code that may be operable to cause a display system 134 to present ultrasound images acquired by the image acquisition processor 140. For example, the positioning processor 150 may be configured to receive the position of the ultrasound probe 104 relative to a target view of a target structure from the ultrasound probe 104, image acquisition processor 140, and/or the probe tracking processor 160, and/or retrieve the location from the archive 138 and/or any suitable data storage medium, in order to cause a display system to update the position of the ultrasound probe 104 and/or the reference target view image on the display 134.

In various embodiments, the positioning processor 150 may comprise suitable logic, circuitry, interfaces, and/or code that may be operable to cause a display system 134 to present a reference target view image 320 with a target view of a selected target structure. For example, the positioning processor 150 may cause the display system to present the reference target view image 320 with a target view at a first position on the display 134 and an ultrasound probe 104 location at a second position on the display 134. In some embodiments, the ultrasound probe 104 location may be represented by ultrasound image 330. In some other embodiments, the ultrasound probe 104 location may be represented by a graphical marker. As the ultrasound probe 104 moves relative to the reference ultrasound image, the positioning processor 150 may continuously update the position of the ultrasound image 330 relative to position of the reference target view image 320 on the display so that a user may receive real-time feedback as the ultrasound probe 104 moves towards a location to acquire the target view from the target structure.

In various embodiments, as the ultrasound probe is moved, the positioning processor 150 may reflect the movements of the ultrasound probe on the display 134. For example, sliding of the ultrasound probe 104 along a main plane (e.g., azimuth direction) would be presented by the positioning processor 150 as a displacement of the ultrasound image 330 on the display 134. In some examples, angular rotations of the ultrasound probe 104 may also be reflected by the positioning processor 150 on the display 134. The positioning processor 150 may render movement of larger distances by the ultrasound probe 104 on the user interface 310 by moving the ultrasound 330 closer to the reference target view image 320 and/or manipulating the ultrasound 330 in size and/or shape to reflect the ultrasound probe movement towards the reference target view image 320. In some examples, the positioning processor 150 may also modify the reference target view image 320 in size and/or shape in order to help reflect movement of the ultrasound image 330 relative to the reference target view image 342, which represents the actual movement of the ultrasound probe 104 towards the target structure.

The position of the reference target view image 320 may remain fixed on the display while the ultrasound image 330 moves towards the target view, the position of ultrasound image 330 may be fixed while the position of the reference target view image is updated on the display 134, and/or both the positions of the ultrasound image and the target view may be changed/updated on the display 134, The positioning processor 150 may receive location information for the ultrasound probe 104 relative to the target view from the ultrasound probe 104, the image acquisition processor 140, and/or the probe tracking processor 160. Additionally and/or alternatively, the positioning processor 150 may provide the position information for the ultrasound image acquisition and the ultrasound probe 104 to the target processor 170 and/or the graphical processor 180. Additionally and/or alternatively, the position information for the ultrasound image 330, the continuous ultrasound image acquisitions, and the reference target view image 320 may be stored at archive 138 and/or any suitable computer readable medium.

In some embodiments, the positioning processor 150 may provide an intermediate target on the display so that a user may move the ultrasound probe 104 to the intermediate target. In some examples, the intermediate target may represent an anatomical structure, or a point in space and time near an anatomical structure. Once the ultrasound probe 104 reaches the anatomical structure and/or point in space and time, the positioning processor 150 may provide additional intermediate targets for the ultrasound probe 104 to reach until the ultrasound probe 104 is sufficiently close to the target structure, in which case the positioning processor 150 may place the reference target view image 320 on the display 134 as the target. Additional visual and/or auditory instructions may be provided to the user on the display 134, such as text or graphical illustrations that may communicate the next movement of the ultrasound probe 104.

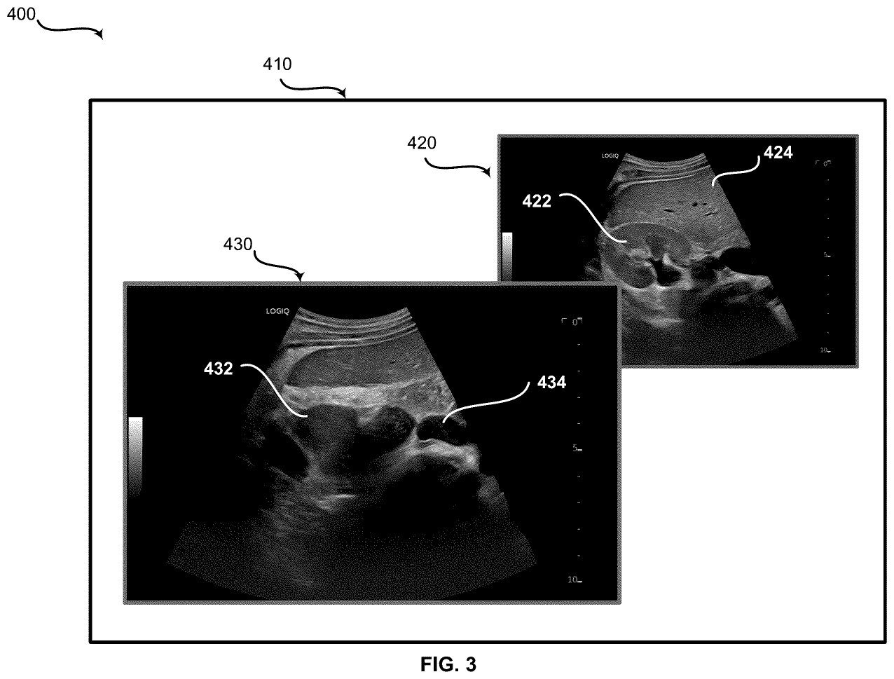

FIG. 3 is a second exemplary display of a user interface 410 for acquiring a target view of an anatomical structure, in accordance with various embodiments. Referring to FIG. 3, the display 400 comprises reference target view image 420 and ultrasound image 430. The reference target view image 420, target view 424, selected target structure 422, ultrasound image view 434, target structure 432, and ultrasound image 430 may be modifiable by user input received via the user input device 130 and/or touchscreen display 130, 134 to adjust a position and/or size of each. The selected target structure 422, 432 may be provided by the image acquisition processor 140 and/or the positioning processor 150 to the graphical processor 180.

FIG. 3 depicts a movement of the ultrasound image 430 towards the reference target view image 420 in comparison to FIG. 2. In some embodiments, as the ultrasound probe 104 moves towards the target structure, the positioning processor 150 causes the ultrasound image 430 on the display 400 to move towards the reference target view image 420. In some other embodiments, the positioning processor 150 causes reference target view image 420 on the display to move towards the ultrasound image 430 on the display 400. Additionally and/or alternatively, the ultrasound image 430 may be an updated ultrasound image 430 from ultrasound image 330 of FIG. 3, updated by the image acquisition processor 140 as the ultrasound probe 104 moves towards the target view of the target structure.

Referring again to FIG. 1, the probe tracking processor 160 may comprise suitable logic, circuitry, interfaces, and/or code that may be operable to track a location of an ultrasound probe 104 (e.g. continuously obtaining the location information of the ultrasound probe 104). For example, as the ultrasound probe 104 is moved by a user, the probe tracking processor 160 tracks the location of the ultrasound probe 104 and provides real-time and/or feedback to the user by providing the location of the ultrasound probe 104 relative to the reference target view image 320, 420 to the positioning processor 150. The probe tracking processor 160 may track ultrasound probe 104 location receiving location and/or movement information from the ultrasound probe, by comparing a location determined from a comparison of an ultrasound image obtained by the image acquisition processor 140 with a reference target view and/or by using artificial intelligence and/or machine learning to identify anatomical structures and the location of the ultrasound probe relative to the anatomical structures in the obtained ultrasound images and/or volume with a high degree of probability. For example, image analysis algorithms, one or more deep neural networks (e.g., a convolutional neural network such as u-net) and/or may utilize any suitable form of image analysis techniques, artificial intelligence, or machine learning processing functionality configured to determine a location based on acquired ultrasound images. Additionally and/or alternatively, the image analysis techniques, artificial intelligence, or machine learning processing functionality configured to determine a location based on ultrasound images may be provided by a different processor or distributed across multiple processors at the ultrasound system 100 and/or a remote processor communicatively coupled to the ultrasound system 100. For example, the location functionality may be provided as a deep neural network that may be made up of, for example, an input layer, an output layer, and one or more hidden layers in between the input and output layers. Each of the layers may be made up of a plurality of processing nodes that may be referred to as neurons. For example, the location functionality may include an input layer having a neuron for each pixel of an ultrasound image and/or voxel of an ultrasound volume. The output layer may have a neuron corresponding to each heart muscle, heart chamber, and/or any suitable anatomical structure. Each neuron of each layer may perform a processing function and pass the processed ultrasound image information to one of a plurality of neurons of a downstream layer for further processing. As an example, neurons of a first layer may learn to recognize edges of structure in the obtained ultrasound image and/or volume. The neurons of a second layer may learn to recognize shapes based on the detected edges from the first layer. The neurons of a third layer may learn positions of the recognized shapes relative to landmarks in the obtained ultrasound image. The processing performed by the deep neural network may identify anatomical structures and the location of the anatomical structures in the obtained ultrasound images with a high degree of probability.

In an exemplary embodiment, the probe tracking processor 160 may track the location of the ultrasound probe 104 and provide the ultrasound probe 104 location information to the positioning processor 150 for display on the display system 134 along with a target view. The ultrasound probe 104 location may be represented by an ultrasound image and/or any graphical marker on the display 134. For example, if the user moves the ultrasound probe 104 towards the target view of the target structure, the ultrasound image acquisition(s) representing the ultrasound probe location move towards the reference ultrasound image. In other examples, if the user moves the ultrasound probe 104 away from the target structure, the ultrasound image acquisition moves away from the reference ultrasound image. The user may produce other movements with the ultrasound probe 104, such as left, right, rotations, sliding, rocking, angling, etc., and the ultrasound image acquisition may move accordingly and provide the user feedback regarding the movement of the ultrasound (e.g., if the ultrasound probe 104 is moving away from, towards, to the left of the target view, to the right of the target view, etc.). Providing the user with real-time and/or continuous feedback regarding the location of the ultrasound probe 104 with regards to the target view allows the user to intuitively move the ultrasound probe 104 in an appropriate direction and/or to an appropriate location in order to reach the target view of the selected target structure.

In some embodiments, the probe tracking processor 160 may store and/or track the locations of the ultrasound probe 104 over time and use the stored locations in order to assess how far the ultrasound probe 104 has traveled, whether the ultrasound probe 104 is moving towards or away from the target structure, and/or to determine how far the ultrasound probe 104 is from the target structure. For example, the probe tracking processor 160 may store relative locations of the ultrasound probe 104 with regards to previous locations, relative location to the target view and/or the target structure, and any location assessments made over time. The probe tracking processor 160 may provide the location information for the ultrasound probe 104 to the positioning processor 150, the target processor 170, and/or the graphical processor 180. Additionally and/or alternatively, the location information for the ultrasound probe 104 location may be stored at archive 138 and/or any suitable computer readable medium.

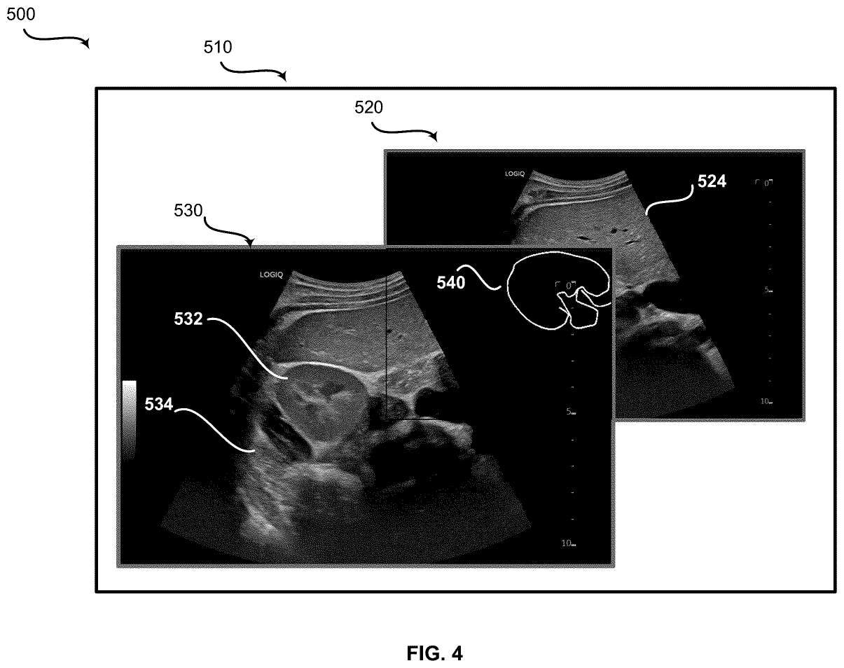

FIG. 4 is a third exemplary display 500 of a user interface 510 for acquiring a target view of an anatomical structure, in accordance with various embodiments. FIG. 4 depicts a movement of the ultrasound image 530 towards the reference target view image 520 in comparison to FIGS. 2 and 3. Referring to FIG. 4, the display 500 comprises a reference target view image 520 and an ultrasound image 530. As the ultrasound probe 104 changes location, the ultrasound image 530 may move with the movement of the probe. For example, as the probe moves towards the reference target view image 520, ultrasound image 530 may also move towards the reference target view image 520, thereby providing a user with continuous feedback towards the reference target view image 520. The image acquisitions may be continuously updated on the display 134 and the position of the ultrasound image 530 may be continuously updated on the screen as the ultrasound probe 104 changes location and moves toward the reference target view image 520. For example, the ultrasound image 530 may be an updated ultrasound image 530 from ultrasound image 330 and 430 of FIGS. 2 and 3, updated by the image acquisition processor 140 as the ultrasound probe 104 moves towards the target view of the target structure. In some embodiments, as the reference target view image 520 and the ultrasound image 530 begin to overlap, the selected target structure 532 may be presented as a graphical outline 540 representing the selected target structure in the reference target view image 520 and/or the ultrasound image 530.

In an exemplary embodiment, the probe tracking processor 160 may track the location of the ultrasound probe 104 and provide the location information to the positioning processor 150, the target processor 170, and/or the graphical processor 180. For example, if the user moves the ultrasound probe 104 towards the target structure, the ultrasound image 530 moves toward the reference target view image 520. In other examples, if the user moves the ultrasound probe 104 away from the target structure, the ultrasound image 530 moves away from the reference target view image 520.

The user may produce other movements with the ultrasound probe 104, such as left, right, rotations, sliding, rocking, angling, etc., and the first ultrasound image 530 may move accordingly and provide the user feedback regarding the movement of the ultrasound (e.g., if the ultrasound probe 104 is moving away from, towards, to the left of the target view, to the right of the target view, etc.). Providing the user with real-time feedback regarding the location of the ultrasound probe 104 with regards to the target view allows the user to intuitively move the ultrasound probe 104 in an appropriate direction and/or to an appropriate location in order to align the ultrasound image 530 with the reference target view image 520 of the target structure 522, 532, which is a representation of the ultrasound probe 104 reaching the target view of the target structure.

Referring to FIG. 1, the graphical processor 180 may comprise suitable logic, circuitry, interfaces and/or code that may be configured to overlay graphical markers on ultrasound images having a target structure or on a reference target view image in response to a user input or automatically based on the information provided by the image acquisition processor 140, the positioning processor 150, the probe tracking processor 160, and/or the target processor 170. For example, the graphical processor 180 may place a graphical marker on ultrasound images, a reference image including a target view, and/or on a display. The graphical marker may be used to identify a selected target structured and/or an ultrasound probe 104 location, for example. The graphical processor 180 may be used to provide real-time location information for ultrasound probe 104 and for the selected target structure in the ultrasound image and/or the target view. The graphical processor 180 may provide a visual indicator as an outline of the selected target structure as depicted in FIGS. 4-6, a different shape of visual indicator, colorized pixels, different graphical effects (e.g., blinking, flashing, pulsing, color-changing, etc.), as non-limiting examples, and/or may be customizable by a user. The graphical marker processor may also output alerts, such as sound, visual indicators, textual indicators, and may change visual indications based on proximity of the ultrasound image to the target view.

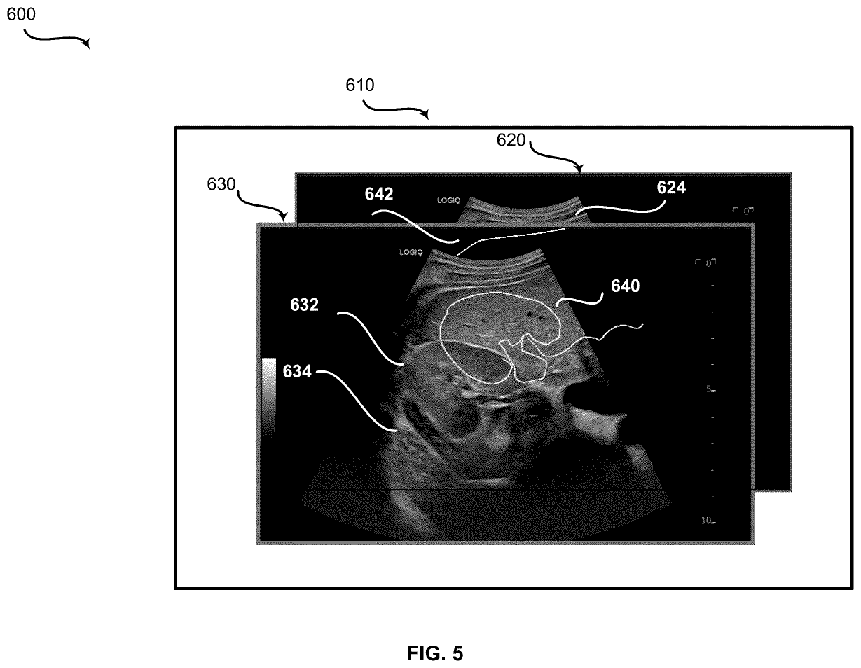

FIG. 5 is a fourth exemplary display 600 of a user interface 610 for acquiring a target view of an anatomical structure, in accordance with various embodiments. FIG. 5 depicts a movement of the ultrasound image 630 towards the reference target view image 620 in comparison to FIGS. 2-4. Referring to FIG. 5, the display 600 comprises a reference target view image 620 and an ultrasound image 630. The selected target structure 640 may be presented as a bounding box, colorized pixels, an outline, and/or any suitable identifier. For example, the selected target structure may be depicted as graphical outline 640 as the ultrasound image 630 moves into alignment with reference target view image 620. The graphical outline 640 may be continuously updated by the graphical processor 180 as the ultrasound image moves as the ultrasound probe 104 is moved by a user.

Although one graphical marker is depicted in FIG. 5, additional graphical markers may be included in the ultrasound images and/or the target view. For example, graphical markers may be placed to denote the target view and/or the ultrasound image, and/or additional structures within the ultrasound image and/or the target view. The graphical processor 180 may receive information from the image acquisition processor 140, the positioning processor 150, the probe tracking processor 160, and/or the target processor 170 in order to update the placement and/or type of graphical marker displayed.

Referring to FIG. 1, a target processor 170 comprises suitable logic, circuitry, interfaces, and/or code that may be operable to determine whether an ultrasound probe has reached a target structure and/or a target view of the target structure. The target processor 170 may be configured to receive ultrasound probe 104 location information (e.g., location in space and time of the probe, location relative to the target view) from the probe tracking processor 170 and/or a target view of the selected target structure from the image acquisition processor 140. Additionally and/or alternatively the ultrasound probe 104 location may be received directly from the ultrasound probe 104.

In some embodiments, the target processor 170 may use the location obtained from the probe tracking processor 170 to determine the proximity of the probe to the target view and/or target structure, the relative location of the ultrasound probe 104 to previous locations, total distance traveled by the ultrasound probe 104, relative location from an intermediate target, etc. The target processor 170 may provide guidance on the display 134 to guide the ultrasound probe 104 towards the target view by providing real-time feedback (e.g. the ultrasound image moving towards the target view).

In some embodiments where the ultrasound probe 104 is further than a threshold distance from the target structure, the target processor 150 may provide an intermediate target on the display so that a user may move the ultrasound probe 104 to the intermediate target and/or may provide an indication to the positioning processor 150 to provide an intermediate target on the display. Once the ultrasound probe 104 reaches the intermediate target, the target processor 170 may determine a next movement for the user and provide additional intermediate targets for the ultrasound probe 104 to reach until the selected target structure is within a threshold distance. In some embodiments, once the target structure is within the threshold distance, a reference target view image may be provided on the display 134 so that the user may move the ultrasound probe 104 towards the reference target view image as described above with reference to FIGS. 1-6. Additional visual and/or auditory instructions may be provided to the user on the display 134, such as text or graphical illustrations that may communicate the next movement of the ultrasound probe 104.

In some embodiments, if the location of probe misses the target view or passes the target view, the target processor 170 may recalculate the proximity of the ultrasound probe 104 to the target view. Additionally, the display 134 may be updated by the positioning processor 150 with the location of the ultrasound probe 104 past the target view to signal to the user that the ultrasound probe 104 has passed the selected target structure and/or the target view.

The target processor 170 may determine whether the location information of the ultrasound probe 104 is near the selected target structure and/or the target view in order to determine whether the ultrasound probe 104 has acquired the target view. In some embodiments, the determination of whether the ultrasound probe has acquired the target view is based on calculating a distance the ultrasound probe 104 has traveled within a period of time (e.g., since the last image acquisition, in the last few minutes, etc.), a total distance that the ultrasound probe 104 has traveled (e.g., since the first image acquisition), or by assessing a quality of the one or more subsequently acquired ultrasound images relative to the target view (e.g. if a majority of the ultrasound image overlaps with the target view, if a majority of the selected target structure in the ultrasound image aligns with the selected target structure in the target view, etc.).

When the ultrasound probe 104 reaches the target view, the target processor 170 may send an output signal. In some examples, the output signal may be a notification, such as a visual (e.g., an alert on the display system 134), auditory (e.g. a sound), and/or physical indicator (e.g. vibration of ultrasound probe 104) to notify a user to pause the ultrasound probe 104 at the current location.

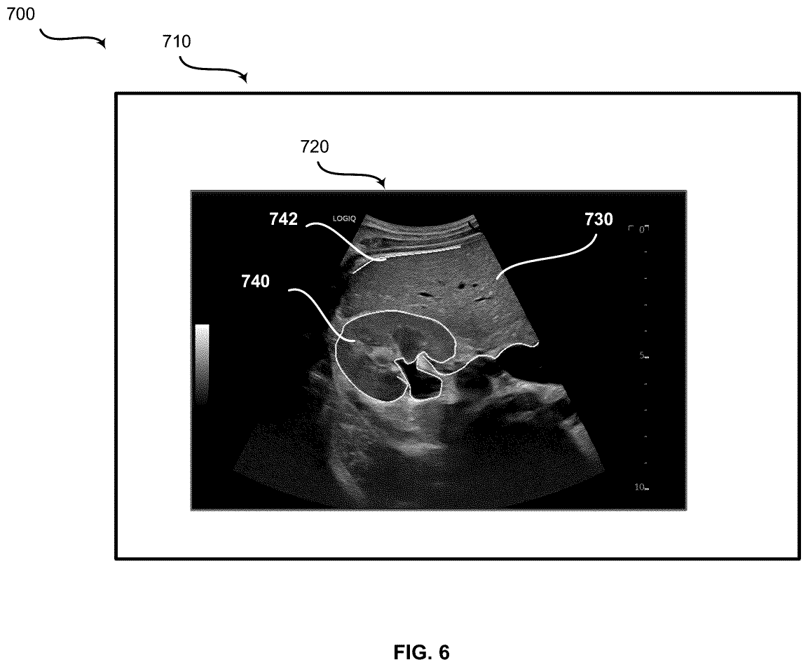

FIG. 6 is a fifth exemplary display 700 for acquiring a target view of an anatomical structure, in accordance with various embodiments. The display 700 comprises an ultrasound image with an acquired target view 720 of a selected target structure 730. The ultrasound image with an acquired target view 720 includes one or more selected target structures 730 that have been aligned with a target view and/or selected target structures in the target view as described above with reference to FIGS. 2-5. As the ultrasound probe 104 moves towards a target view of a selected target structure, the location of the ultrasound probe 104 is tracked by the probe tracking processor 160. Once the ultrasound probe 104 reaches the target view, the target processor 170 may output a signal indicating the target view has been reached. In some examples, the output signal may be a notification, such as a visual (e.g., an alert on the display system 134), auditory (e.g. a sound), and/or physical indicator (e.g. vibration of ultrasound probe 104) to notify a user to pause the ultrasound probe 104 at the current location. Additionally, or alternatively, the graphical processor 180 may provide a visual indicator, colorized pixels, different graphical effects (e.g., blinking, flashing, pulsing, color-changing, etc.), text, as non-limiting examples, to indicate to the user that the target view has been reached.

Referring again to FIG. 1, the display system 134 may be any device capable of communicating visual information to a user. For example, a display system 134 may include a liquid crystal display, a light emitting diode display, and/or any suitable display or displays. The display system 134 can be operable to present 2D/3D ultrasound images 320, 330, 420, 430, 520, 530, 620, 630, 720, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things.

In various embodiments, the archive 138 stores 2D ultrasound images 320, 330, 420, 430, 520, 530, 620, 630, 720, rendered 3D/4D volumes, instructions for automatically detecting and tracking target structures 322, 332, 422, 432, 532, 632, and other anatomical structures 642, 742, instructions for causing a display system 134 to present 2D ultrasound images 320, 330, 420, 430, 520, 530, 620, 630, 720 and for triggering additional ultrasound image acquisitions, instructions for identifying a target view 324, 334, 424, 434, 524, 534, 624, 634 and tracking an ultrasound probe 104 to obtain the acquired target view 730, instructions for overlaying ultrasound images 320, 330, 420, 430, 520, 530, 620, 630, 720, with graphical markers 540 640, 740 and dynamically updating the graphical markers 540 640, 740 on the ultrasound images 320, 330, 420, 430, 520, 530, 620, 630, 720 over time, instructions for outputting a signal when the acquired target view 730 is attained.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

Still referring to FIG. 1, the training system 200 may comprise a training engine 210 and a training database 220. The training engine 210 may comprise suitable logic, circuitry, interfaces and/or code that may be operable to train the neurons of the deep neural network(s) (e.g., artificial intelligence model(s)) inferenced (i.e., deployed) by the image acquisition processor 140, the positioning processor 150, the probe tracking processor 160, the target processor 170, and/or graphical processor 180. For example, the artificial intelligence model inferenced by the image acquisition processor 140 may be trained to automatically identify anatomical structures depicted in an ultrasound image and/or volume using database(s) 220 of classified ultrasound images of anatomical structures. As another example, the artificial intelligence model inferenced by the image acquisition processor 140, positioning processor 150, probe tracking processor 160, and/or target processor 170 may be trained to automatically identify target structures, surrounding structures, target structure shapes, major/minor axes of target structures, and the like depicted in an ultrasound volume using database(s) 220 of classified ultrasound volumes of possible target structures.

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the ultrasound system 100 as shown in FIG. 1. Additionally and/or alternatively, components or all of the training system 200 may be integrated with the ultrasound system 100 in various forms. In some examples, the training image databases may include atlases (e.g., labeled maps of anatomical structures, original images, segmentation masks, as non-limiting examples). In some examples, the training image databases 220 may be integrated with the archive 138 or vice versa.

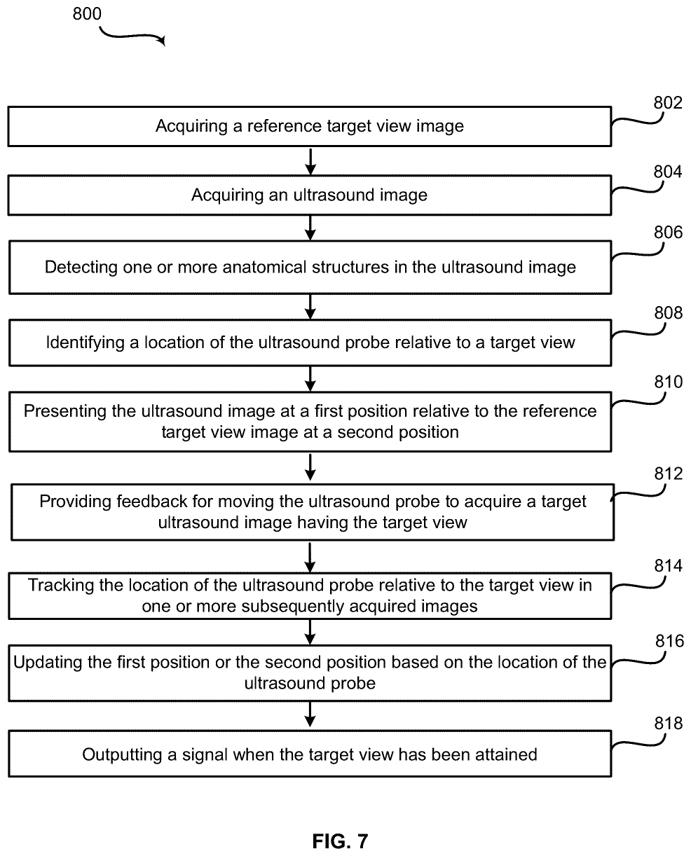

FIG. 7 is a flow chart 800 illustrating exemplary steps 802-816 that may be utilized for providing a continuous guidance user interface for acquiring a target view, in accordance with various embodiments. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 802, a signal processor 132 of the ultrasound system 100 may be configured to determine a target view 324, 334, 424, 434, 524, 534, 624, 634 of a reference target view image 320, 420, 520, 620. For example, an image acquisition processor 140 may be configured to receive a user input selecting a target view of a target structure 322, 422, such as a cardiac structure, a gastroenterological structure, a urological structure, a reproductive structure, a cardiac structure, a pulmonary structure, and/or any suitable anatomical structures, as non-limiting examples, via a user input device 130. The image acquisition processor 140 may acquire first ultrasound image(s) 330 in response to the selection of a target structure 322, 422.

At step 804, an ultrasound probe 104 of an ultrasound system 100 performs an initial ultrasound image acquisition as ultrasound image 330. The acquired first ultrasound images 330 of the initial ultrasound image acquisition may be provided to the image acquisition processor 140 and/or stored at archive 138 and/or any suitable computer readable medium.

At step 806, the signal processor 132 of the ultrasound system 100 identifies a location of the ultrasound probe 104 relative to the target view 324, 334, 424, 434, 524, 534, 624, 634 of the target structure 322, 422. For example, a probe tracking processor 160 of the signal processor 132 may be configured to obtain the location of the ultrasound probe 104 and provide the location of the ultrasound probe 104 to the positioning processor 150 and/or the target processor 170. The positioning processor 150 may be configured to receive from the image acquisition processor 140, or retrieve from the archive 138 and/or any suitable data storage medium, the identity and location of a selected target structure 322, 422. The positioning processor 150 may be configured to identify the localized selected target structure by overlaying a marker, a bounding box, colorizing pixels, outline and/or any suitable identification technique.

At step 808, the signal processor 13 of the ultrasound system 100 may present the ultrasound image 330, 430, 530, 630 at a first position on the display 134 relative to the reference target view image 320, 420, 520, 620 at a second position on the display 134. For example, a graphical processor 180 may be configured to indicate the selected target structure 322, 332, 422, 432, 532, 632, on a display using a graphical marker 540, 640, 740. The selected target structure 322, 422 location may be determined automatically by the positioning processor 150 using information obtained from the image acquisition processor 140 and/or the ultrasound probe 104 location obtained from the probe tracking processor 160. The positioning processor 150 may provide the anatomical structure location to the target processor 170 and/or the graphical processor 180 and may provide real-time updates of the anatomical structure location at the display system 134.

At step 810, the signal processor 132 may provide feedback for moving the ultrasound probe 104 acquiring ultrasound images 330, 430, 530, 630 to acquire a reference target view image 320, 420, 520. As the ultrasound probe 104 moves relative to the reference target view image 320, 420, 520, the positioning processor 150 may continuously update the position of the ultrasound image 330, 430, 530, 630 relative to the target view 330, 430, 530, 630 so that a user may receive real-time feedback as the ultrasound probe 104 moves into alignment with the reference target view image 320, 420, 520. Additionally and/or alternative the real-time feedback may include a notification, such as a visual (e.g., an alert on the display system 134), auditory (e.g. a sound), and/or physical indicator (e.g. vibration of ultrasound probe 104).

At step 812, the signal processor 132 may track the location of the ultrasound probe 104 relative to the reference target view image 320, 420, 520 in one or more subsequently acquired images 430, 530, 630. The probe tracking processor 160 of the signal processor 132 provides real-time location information of the ultrasound probe 104 to the positioning processor 150 in order to update the position of the reference target image 320, 420, 520, 620 and/or the ultrasound image 330, 430, 530, 630 on the display 134.

At step 814, the signal processor 132 updates the first position and/or the second position based on the location of the ultrasound probe 104. As the ultrasound probe 104 moves relative to the reference target view image 320, 420, 520, the positioning processor 150 may continuously update the position of the ultrasound image 330, 430, 530, 630 relative to the reference target view image 320, 420, 520, 620 and/or the position of the ultrasound image 330, 430, 530, 630 relative to the reference target view image 320, 420, 520, 620 so that a user may receive real-time feedback as the ultrasound probe 104 moves into alignment with the reference target view image 320, 420, 520, 620. The position of the reference target view image 320, 420, 520, 620 may remain fixed on the display while the ultrasound image 330, 430, 530, 630 moves towards the reference target view image 320, 420, 520, 620, the position of ultrasound image 330, 430, 530, 630 may be fixed while the position of the reference target view image 320, 420, 520, 620 is updated on the display 134, and/or both the positions of the ultrasound image 330, 430, 530, 630 and the reference target view image 320, 420, 520, 620 may be changed/updated.

At step 816, the signal processor 132 may output a signal when the acquired target view 740 has been attained by the ultrasound probe 104. In some examples, the output signal may be a notification, such as a visual (e.g., an alert on the display system 134), auditory (e.g. a sound), and/or physical indicator (e.g. vibration of ultrasound probe 104) to notify a user to pause the ultrasound probe 104 at the current location.

Aspects of the present disclosure provide a method 800 and system 100 for acquiring a target ultrasound image 720 having a target view 324, 424, 524, 624 of one or more anatomical structures 322, 332, 422, 432, 532, 632 including acquiring, by an ultrasound probe 104 of an ultrasound system 100, an ultrasound image 330, 430, 530, 630. The method 800 may include processing, by the at least one processor 132, 160, the ultrasound image 330, 430, 530, 630 to identify a location of the ultrasound probe 104 relative to the reference target view image 320, 420, 520, 620 of the one or more anatomical structures 322, 332, 422, 432, 532, 632. The method 800 may comprise causing, by the at least one processor 132, 150, a display system to present the ultrasound image 330, 430, 530, 630 and the reference target view image 320, 420, 520, wherein the ultrasound image 320, 420, 520 is presented at a first position on the display system relative to a second position on the display system of the reference target view image 320, 420, 520, and wherein the first position and the second position provide feedback for moving the ultrasound probe 104 to acquire the target ultrasound image 720 having the target view.

The method 800 may comprise tracking, by the at least one processor 132, 160, the location of the ultrasound probe 104 relative to the reference target view image 320, 420, 520 in one or more subsequently acquired ultrasound images 430, 530, 630. The method 800 may include updating, by the at least one processor 132, 150, the first position or the second position on the display system 134 of the ultrasound image based on the location of the ultrasound probe 104.

In an exemplary embodiment, the method 800 comprises obtaining the target ultrasound image 720 having the target view 730 by aligning the ultrasound image 330, 430, 530, 630 and the reference target view image 320, 420, 520, 620 on the display system. In an exemplary embodiment, the method 800 comprises assessing, by the at least one processor 132, 170, whether the ultrasound probe 104 has obtained the target view 324, 424, 524, 624, 730 by: calculating a distance the ultrasound probe 104 has traveled within a period of time, calculating a total distance that the ultrasound probe 104 has traveled, or assessing a quality of the one or more subsequently acquired ultrasound images 330, 430, 530, 630 relative to the target view 324, 424, 524, 624.

In an exemplary embodiment, the method 800 comprises overlaying, by the at least one processor 132, 180, the ultrasound image 330, 430, 530, 630 with a graphical representation of the one or more anatomical structures 322, 332, 422, 432, 532, 632 in the ultrasound image 330, 430, 530, 630, and updating, by the at least one processor 132, 180, the graphical representation 540, 640, 642, 740, 742 of the one or more anatomical structures in the ultrasound image 330, 430, 530, 630, 720 and the first position on the display system 134 based on the location of the ultrasound probe 104 with respect to the target view 324, 424, 524, 624.

In an exemplary embodiment, the method 800 comprises aligning, by the at least one processor 132, 150, 180 the graphical representation of the one or more anatomical structures 322, 332, 422, 432, 532, 632 in the ultrasound image 330, 430, 530, 630, 720 with the reference target view image 320, 420, 520, 620. In an exemplary embodiment, the method 800 comprises outputting, by the at least one processor 132, 170, a signal when the target view 324, 424, 524, 624 has been attained. In an exemplary embodiment, the ultrasound image 330, 430, 530, 630 is a 2D or 3D image.

Various embodiments provide an ultrasound system 100 for acquiring a target ultrasound image 720 having a target view 730 of one or more anatomical structures 322, 332, 422, 432, 532, 632 comprising an ultrasound probe 104 configured to process the ultrasound image 330, 430, 530, 630, 720 to identify a location of the ultrasound probe 104 relative to the target view 324, 424, 524, 624 of the one or more anatomical structures 322, 332, 422, 432, 532, 632.

The at least one processor 132, 150 may be configured to cause a display system 134 to present the ultrasound image 330, 430, 530, 630, 720 and the reference target view image 330, 430, 530, 630, wherein the ultrasound image 330, 430, 530, 630, 720 is presented at a first position on the display system 134 relative to a second position on the display system 134 of the reference target view image 320, 420, 520, 620, and wherein the first position and the second position provide feedback for moving the ultrasound probe 104 to acquire the target ultrasound image 720 having the target view 730. The at least one processor 132, 160 may be configured to track the location of the ultrasound probe 104 relative to the target view 324, 424, 524, 624 in one or more subsequently acquired ultrasound images 430, 530, 630, 720. The at least one processor 132, 150 may be configured to update the first position on the display system 134 of the ultrasound image 330, 430, 530, 630, 720 based on the location of the ultrasound probe 104.

In a representative embodiment, the at least one processor 132, 150 is further configured to obtain the target ultrasound image 720 having the target view 730 by aligning the ultrasound image 330, 430, 530, 630, 720 and the reference target view image 320, 420, 520, 620 on the display system. In a representative embodiment, the at least one processor 132, 170 may be configured to assess whether the ultrasound probe 104 has obtained the target view 324, 424, 524, 624 by calculating a distance the ultrasound probe 104 has traveled within a period of time, a total distance that the ultrasound probe 104 has traveled, or by assessing a quality of the one or more subsequently acquired ultrasound images 430, 530, 630, 720 relative to the target view 324, 424, 524, 624.

In a representative embodiment, the at least one processor 132, 180 is further configured to overlay the ultrasound image 330, 430, 530, 630 with a graphical representation of the one or more anatomical structures 322, 332, 422, 432, 532, 632 in the ultrasound image 330, 430, 530, 630, 720, and wherein the at least one processor 132, 180 is further configured to update the graphical representation 540, 640, 642, 740, 742 of the one or more anatomical structures 322, 332, 422, 432, 532, 632 in the ultrasound image 330, 430, 530, 630, 720 and the first position on the display system 134 based on the location of the ultrasound probe 104 with respect to the target view 324, 424, 524, 624.

In a representative embodiment, the at least one processor 132, 150, 180 may be configured to align the graphical representation 540, 640, 642, 740, 742 of the one or more anatomical structures 322, 332, 422, 432, 532, 632 in the ultrasound image 330, 430, 530, 630, 720 with the reference target view image 320, 420, 520, 620. In a representative embodiment, the at least one processor 132, 170 may be configured to output a signal when the target view 324, 424, 524, 624, has been attained.

Various embodiments provide an ultrasound system 100 for acquiring a target ultrasound image 720 having a target view 730 of one or more anatomical structures 322, 332, 422, 432, 532, 632 comprising an ultrasound probe 104 configured to acquire an ultrasound image 330, 430, 530, 630, at least one processor 132, 140 configured to process the ultrasound image 330, 430, 530, 630, 720 to identify a location of the ultrasound probe 104 relative to the target view 324, 424, 524, 624 of the one or more anatomical structures 322, 332, 422, 432, 532, 632.

The at least one processor 132, 150 may be configured to cause a display system 134 to present the ultrasound image 330, 430, 530, 630, 720 and the reference target view image 330, 430, 530, 630, wherein the ultrasound image 330, 430, 530, 630, 720 is presented at a first position on the display system 134 relative to a second position on the display system 134 of the reference target view image 320, 420, 520, 620, and wherein the first position and the second position provide feedback for moving the ultrasound probe 104 to acquire the target ultrasound image 720 having the target view 730. The at least one processor 132, 160 may be configured to track the location of the ultrasound probe 104 relative to the target view 324, 424, 524, 624 in one or more subsequently acquired ultrasound images 430, 530, 630, 720. The at least one processor 132, 150 may be configured to update the first position on the display system 134 of the ultrasound image 330, 430, 530, 630, 720 based on the location of the ultrasound probe 104.

The at least one processor 132, 170 may be configured to assess whether the ultrasound probe 104 has obtained the target view 324, 424, 524, 624 by calculating a distance the ultrasound probe 104 has traveled within a period of time, a total distance that the ultrasound probe 104 has traveled, or by assessing a quality of the one or more subsequently acquired ultrasound images 430, 530, 630, 720 relative to the target view 324, 424, 524, 624.

In a representative embodiment, the at least one processor 132, 150 may be configured to align the ultrasound image 330, 430, 530, 630, 720 and the reference target view image 320, 420, 520, 620 on the display system when the target view 324, 424, 524, 624 is attained.

In a representative embodiment, the at least one processor 132, 180 is further configured to overlay the ultrasound image 330, 430, 530, 630 with a graphical representation of the one or more anatomical structures 322, 332, 422, 432, 532, 632 in the ultrasound image 330, 430, 530, 630, 720, and wherein the at least one processor 132, 180 is further configured to update the graphical representation 540, 640, 642, 740, 742 of the one or more anatomical structures 322, 332, 422, 432, 532, 632 in the ultrasound image 330, 430, 530, 630, 720 and the first position on the display system 134 based on the location of the ultrasound probe 104 with respect to the target view 324, 424, 524, 624.

In a representative embodiment, the at least one processor 132, 150, 180 may be configured to align the graphical representation 540, 640, 642, 740, 742 of the one or more anatomical structures 322, 332, 422, 432, 532, 632 in the ultrasound image 330, 430, 530, 630, 720 with the reference target view image 320, 420, 520, 620. In a representative embodiment, the at least one processor 132, 170 may be configured to provide visual or auditory guidance to a user in order to acquire the target view 324, 424, 524, 624. In a representative embodiment, the at least one processor 132, 170 may be configured to output a signal when the target view 324, 424, 524, 624, has been attained. In a representative embodiment, the ultrasound image is a 2D or 3D image.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for acquiring a target ultrasound image having a target view of one or more anatomical structures.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method for acquiring a target ultrasound image having a target view of one or more anatomical structures comprising:
acquiring, by an ultrasound probe of an ultrasound system, an ultrasound image;
processing, by at least one processor, the ultrasound image to identify a location of the ultrasound probe relative to the target view of the one or more anatomical structures;
causing, by the at least one processor, a display system to present the ultrasound image and a reference target view image, wherein the ultrasound image is presented at a first position on the display system relative to a second position on the display system of the reference target view image, and wherein the first position and the second position provide feedback for moving the ultrasound probe to acquire a target ultrasound image having the target view;
tracking, by the at least one processor, the location of the ultrasound probe relative to the target view in one or more subsequently acquired ultrasound images; and
updating, by the at least one processor, the first position or the second position on the display system of the ultrasound image based on the location of the ultrasound probe.

2. The method of claim 1, further comprising obtaining, by the at least one processor, the target ultrasound image having the target view by aligning the ultrasound image and the reference target view image on the display system.

3. The method of claim 1, further comprising assessing, by the at least one processor, whether the ultrasound probe has obtained the target view by: calculating a distance the ultrasound probe has traveled within a period of time, calculating a total distance that the ultrasound probe has traveled, or assessing a quality of the one or more subsequently acquired ultrasound images relative to the target view.

4. The method of claim 1, further comprising overlaying, by the at least one processor, the ultrasound image with a graphical representation of the one or more anatomical structures in the ultrasound image, and updating, by the at least one processor, the graphical representation of the one or more anatomical structures in the ultrasound image and the first position on the display system based on the location of the ultrasound probe with respect to the target view.

5. The method of claim 4, further comprising aligning, by the at least one processor, the graphical representation of the one or more anatomical structures in the ultrasound image with the reference target view image.

6. The method of claim 1, further comprising outputting, by the at least one processor, a signal when the target view has been attained.

7. The method of claim 1, wherein the ultrasound image is a 2D or 3D image.

8. An ultrasound system for acquiring a target ultrasound image having a target view of one or more anatomical structures comprising:
an ultrasound probe configured to acquire an ultrasound image;
at least one processor configured to:
process the ultrasound image to identify a location of the ultrasound probe relative to the target view of the one or more anatomical structures;
cause a display system to present the ultrasound image and a reference target view image, wherein the ultrasound image is presented at a first position on the display system relative to a second position on the display system of the reference target view image, and wherein the first position and the second position provide feedback for moving the ultrasound probe to acquire the target ultrasound image having the target view;
track the location of the ultrasound probe relative to the target view in one or more subsequently acquired ultrasound images; and
update the first position on the display system of the ultrasound image based on the location of the ultrasound probe.

9. The ultrasound system of claim 8, wherein the at least one processor is further configured to obtain the target ultrasound image having the target view by aligning the ultrasound image and the reference target view image on the display system.

10. The ultrasound system of claim 8, wherein the at least one processor is further configured to assess whether the ultrasound probe has obtained the target view by calculating a distance the ultrasound probe has traveled within a period of time, a total distance that the ultrasound probe has traveled, or by assessing a quality of the one or more subsequently acquired ultrasound images relative to the target view.

11. The ultrasound system of claim 8, wherein the at least one processor is further configured to overlay the ultrasound image with a graphical representation of the one or more anatomical structures in the ultrasound image, and wherein the at least one processor is further configured to update the graphical representation of the one or more anatomical structures in the ultrasound image and the first position on the display system based on the location of the ultrasound probe with respect to the target view.

12. The ultrasound system of claim 11, wherein the at least one processor is further configured to align the graphical representation of the one or more anatomical structures in the ultrasound image with the reference target view image.

13. The ultrasound system of claim 8, wherein the at least one processor is further configured to output a signal when the target view has been attained.

14. An ultrasound system for acquiring a target ultrasound image having a target view of one or more anatomical structures comprising:
an ultrasound probe configured to acquire an ultrasound image;
at least one processor configured to:
process the ultrasound image to identify a location of the ultrasound probe relative to the target view of the one or more anatomical structures;
cause a display system to present the ultrasound image and a reference target view image or an intermediate target, wherein the ultrasound image is presented at a first position on the display system relative to a second position on the display system of the reference target view image or the intermediate target, and wherein the first position and the second position provide feedback for moving the ultrasound probe to acquire the target ultrasound image having the target view;
track the location of the ultrasound probe relative to the target view in one or more subsequently acquired ultrasound images;
update the first position of the ultrasound image on the display system based on the location of the ultrasound probe; and
assess whether the intermediate target or the target view has been acquired by calculating a distance the ultrasound probe has traveled within a period of time, a total distance that the ultrasound probe has traveled, or by assessing a quality of the one or more subsequently acquired ultrasound images relative to the target view.

15. The ultrasound system of claim 14, wherein the at least one processor is further configured to align the ultrasound image and the reference target view image or the intermediate target on the display system when the target view has been acquired.

16. The ultrasound system of claim 14, wherein the at least one processor is further configured to overlay the ultrasound image with a graphical representation of the one or more anatomical structures in the ultrasound image and update the graphical representation of the one or more anatomical structures in the ultrasound image on the display system based on the location of the ultrasound probe with respect to the target view.

17. The ultrasound system of claim 16, wherein the at least one processor is further configured to align the graphical representation of the one or more anatomical structures in the ultrasound image with the reference target view image.

18. The ultrasound system of claim 14, wherein the at least one processor is further configured to provide visual or auditory guidance to a user in order to acquire the target view.

19. The ultrasound system of claim 14, wherein the at least one processor is further configured to output a signal when the target view has been attained.

20. The ultrasound system of claim 14, wherein the ultrasound image is a 2D or 3D image.
